(19) United States
(12) Patent Application Publication
(10) Pub. No.: US 2005/0196478 A1
(43) Pub. Date: Sep. 8, 2005

(54) Methods using Lycium barbarum extracts as neuroprotective agents for retinal ganglion cells degeneration

(75) Inventors: Kwok-Fai So, Hong Kong (CN); Wai-Hung Yuen, Hong Kong (CN); Raymond Chuen-Chung Chang, Hong Kong (CN); S. Y. Zee, Hong Kong (CN)

Correspondence Address: DICKSTEIN SHAPIRO MORIN & OSHINSKY LLP, 1177 AVENUE OF THE AMERICAS (6TH AVENUE), 41 ST FL., NEW YORK, NY, 10036-2714, US

(73) Assignee: The University of Hong Kong

(21) Appl. No.: 11/066,628

(22) Filed: Feb. 25, 2005

Related U.S. Application Data
(60) Provisional application No. 60/547,061, filed on Feb. 25, 2004.

(51) Int. Cl. A61K 35/78

(52) U.S. Cl. 424/769

(57) ABSTRACT

An Lycium barbarun extract demonstrates a neuroprotective effect on damaged retinal ganglion cells, preventing and preserving retinal ganglion cells from degeneration in the treated subjects after chronic and traumatic neuronal injury or glaucoma. Compositions include an effective amount of an agent and a pharmaceutically acceptable vehicle.

### CROSS-REFERENCE TO RELATED APPLICATIONS

This application is a non-provisional based on U.S. Provisional Application Ser. No. 60/547,061, filed Feb. 25, 2004, which is incorporated herein in its entirety by reference.

### FIELD OF THE INVENTION

The present invention relates to the methods of producing an active fraction from Lycium barbarum as an effective agent to reduce the death of damaged or undamaged retinal ganglion cells from traumatic injury in mammals. In another aspect, the present invention relates to methods for the protection of retinal ganglion cells following chronic injury in mammals. In yet another aspect, the compositions are used for the treatment of diseases related to cellular damage, including glaucoma and other neurodegenerative diseases.

### BACKGROUND OF THE INVENTION

Glaucomatous optic neuropathy is one of the leading causes of irreversible blindness in the world, the second most common cause of irreversible blindness in U.S. and the most common cause of blindness among blacks. It was estimated in 1966 that nearly 66.8 million persons have primary glaucoma in the world, and 6.7 million persons will suffer from bilateral blindness by the year 2000 (Quigley, 1996; Quigley & Pease, 1996). According to an estimation prepared by the World Health Organization (WHO) in 1997, total number of suspected cases of glaucoma was about 105 million. According to a statistics prepared by Foster and Johnson in 2001, 9.4 million people aged 40 years and older in China was estimated to have glaucomatous optic neuropathy. Of this number, 5.2 million (55%) were blind in at least one eye and 1.7 million (18.1%) were blind in both eyes. This neuropathy reduces vision gradually often without symptoms and therefore individuals having glaucoma were not identified during early stage of glaucoma and before they become blind irreversibly. Visual field loss in glaucoma can be prevented if the disease is detected and treated at early stage.

The major pathological features of glaucoma are the death of retinal ganglion cells (RGSs), cupping and atrophy of optic nerve head leading to the loss of vision. (Leske, 1983; Osborne et al, 1999; Quigley & Green, 1979). RGCs extend their axons through the optic nerve to the visual cortex via the lateral geniculate nucleus in the thalamus (midbrain) (Frost et al, 1979; So et al, 1978 and 1985; Woo et al, 1985). Similar to other neurons in the central nervous system (CNS), RGCs fail to regenerate once they are damaged. Therefore, it is very important to prevent the degeneration of RGCs in any kind of eye diseases, including glaucoma.

The death of retinal ganglion cells in glaucomatous patients is often caused by the elevation of intraocular pressure, although it is not necessary for the progression for the disease (Sarfarazi, 1997). Therefore, all current therapeutic methods for glaucomatous patients target lowering intraocular pressure (IOP). Despite their widespread use in the treatment of glaucomatous optic neuropathy, however, ocular hypotensive agents are not effective in treating a large percentage of people with glaucomatous optic neuropathy. Many people with glaucomatous optic neuropathy have a normal IOP. From 30-50% of people with open angle glaucoma do not initially have ocular hypertension, and as many as 15-50% of patients with glaucomatous optic neuropathy do not have elevated IOP. The absence of increased IOP in certain glaucomatous optic neuropathy patients suggests that there is at least one mechanism other than elevated intraocular pressure that contributes to the optic neuropathy associated with glaucomatous optic neuropathy (Levin, Current Opinion in Ophthalmology 8:9-15, 1997; Levin, Mediguide to Ophthalmology 8:1-5, 1999). Therefore, a considerable effort has been directed toward developing suitable methods for treating glaucomatous optic neuropathy in patients with normal or high IOP, as well as for treating several other optic neuropathies that are not associated with increased IOP.

All the approaches mentioned previously can delay the progressive loss of RGCs but cannot prevent the death of these neurons. While some neuroprotective agents are available, there is still a great need for additional compounds that would be more effective in preventing loss of RGCs from different eye diseases including glaucoma.

Knowledge of mechanisms responsible for natural and experimental optic neuropathy, including axonal transection, optic nerve crush and optic nerve ischemia, may facilitate development of suitable treatments for glaucomatous optic neuropathy and other optic neuropathies affecting the axons of retinal ganglion cells, including ischemic optic neuropathy, inflammatory optic neuropathy, compressive optic neuropathy, and traumatic optic neuropathy. Each of these conditions likely causes apoptosis. The mechanism responsible for initiating apoptosis in retinal ganglion cells has not been unequivocally established. However, it is speculated that decreased retrograde transport of neurotropic factors, decreased levels of endogenous ocular neurotrophins, or any one of several other mechanisms may trigger apoptosis.

A number of animal models have been established to mimic the pathogenic conditions in glaucoma, including ocular hypertension, ocular ischemia and optic nerve transection. Transection of the optic nerve has long been used as an animal model to study the survival and regeneration of RGCs (Cheung et al, 2002; Cho et al, 1999 and 2001; Lu et al, 2003; You et al, 2002). This traumatic injury to the RGCs discontinues the connection between the retina and the brain, resulting in permanent loss of vision. Understanding the mechanisms for the prevention of irreversible loss of RGCs by this model is beneficial to the development of new therapeutic intervention against different eye diseases including glaucoma.

Since an elevated intraocular pressure (IOP) is one of the risk factor in glaucoma, new animal models using monkey or rodent based on ocular hypertension have been developed (Garcia-Valenzuela et al, 1995; Laquis et al, 1998; McKinnon et al, 2002; Mittag et al, 2000; Morrison et al, 1997; Sawada and Neufeld, 1999; Ueda et al, 1998). These models include injection of hypertensive saline, cauterization of episcleral veins, laser photocoagulation on trabecular meshwork, injection of S-antigen and laser photocoagulation to the limbal and episcleral veins. In these studies, an ocular hypertensive model of photocoagulation to the limbal and episcleral veins using argon laser has been employed (Ji et al, 2004; WoldeMussie et al, 2001 and 2002).

Lycium barbarum, a small red berry, is commonly used as traditional Chinese food in home cooking because of its flavour and the general health benefits. It is also used as a herbal medicine for the therapy of a number of eye diseases (Chinese Herbal Medicine Company, 1994; Lam and But, 1999). The red color of the berries is constituted by carotenoid in which only zeaxanthin is present in human macular. Although Lycium barbarum has been widely used in China for centuries with an expected benefit to the visual system, the underlying mechanism of its effect is still not known.

The present inventors recognized the possibility of using such activity and conducted the necessary investigation into that possibility. They found that the aqueous extract isolated from Lycium barbarum could prevent degeneration of RGCs from different kinds of eye diseases.

### BRIEF SUMMARY OF THE INVENTION

The present invention provides a method for reducing RGC death in a subject comprising the steps of:
(a) providing an amount of the effective extract or of an active fraction thereof; and
(b) delivering a therapeutically effective amount of the extract from step (a) to at least a portion of the subject's RGC.

In a preferred embodiment, the extract of step (a) is from Lycium barbarum.

Most conveniently, the effective extract of step (a) is delivered by oral, topical or injection administration, wherein the compound is supplied as a pharmaceutical formulation comprising the effective extract in a therapeutically effective concentration and a pharmaceutically acceptable carrier that may suitably be administered to the subject orally.

Another aspect of the invention is a pharmaceutical formulation for reducing RGC death in a subject by the method of the present invention, the formulation comprising an amount of the effective extract or of an active fraction from Lycium barbarum; and pharmaceutically acceptable salts thereof; in a therapeutically effective concentration and a pharmaceutically acceptable carrier.

It is an object of the invention to provide a method for reducing RGC death in a subject susceptible to an increased rate of RGC.

It is a further object of the invention to provide a pharmaceutical formulation for use in reducing RGC death in a subject susceptible to increased RGC death.

It is an advantage that the extract of Lycium barbarum has already been shown to be relatively safe and nontoxic for humans in other clinical indications.

Additional objects, advantages, and features of the invention will become apparent upon review of the instant specification.

### BRIEF DESCRIPTION OF THE DRAWINGS

FIG. 1 demonstrates the neuroprotective effective of different fractions extracted from Lycium barbarum on RGCs after optic nerve transection in adult hamsters (6-8 weeks, 60-80 g).

FIG. 2 illustrates the protective effect of LBE2 on the survival of RGCs following optic nerve transection.

FIG. 3 shows the loss of RGCs in Spraque-Dawley rats (10-12 weeks, 250-280 g) having ocular hypertension.

FIG. 4 shows intraocular pressure (IOP) in SD rats receiving the LBE2 after laser photocoagulation.

FIG. 5 illustrates the change in body weight of adult hamsters (6-8 weeks, 60-80 g) before and after feeding with LBE2 in an optic nerve transection model as chronic toxicity test. Body weight of hamsters was determined before and 7 days after injury.

FIG. 6 indicates the change in body weight of Spraque-Dawley rats (10-12 weeks, 250-280 g) before and after feeding with LBE2 in an ocular hypertensive model as chronic toxicity test. Body weight of rats was determined before laser photocoagulation.

FIG. 7 shows the change of body weight of young Spraque-Dawley rats (3-5 weeks). Rats were fed with 10 g/kg of LBE2 for 2 weeks (n=8 in each group). The change of body weight of rats before and after oral administration of LBE2 was recorded.

FIG. 8 demonstrates the change of body weight of Spraque-Dawley rats (10-12 weeks). Rats were fed with different dosages of LBE2 for 2 weeks (n=8 in each group). The change of body weight of rats before and after oral administration of LBE2 was recorded. No dead of animal was recorded.

### DETAILED DESCRIPTION OF THE INVENTION

The activity of extracts of this invention, including LBE2, on neuroprotection was investigated through daily oral administration to hamsters following optic nerve transection and by examining the effect of LBE2 on protecting RGCs in ocular hypertensive Spraque-Dawley rats.

The present invention was achieved as a consequence of the following investigations:
examining the effect of four different extracts from Lycium barbarum on damaged RGCs in hamsters following optic nerve transection, to confirm that LBE2 of Lycium barbarum exerted the maximum neuroprotection on RGCs;
examining the neuroprotection of damaged RGCs by oral administration of different dosage LBE2 after optic nerve transection in hamsters, to verify the effect of LBE2 on the survival of RGCs following traumatic injury;
observing the percentage of RGCs loss in LBE2 treated Spraque-Dawley rats having ocular hypertension, and comparing the same to a control group, to verify the neuroprotective effect of LBE2;
comparing the change in intraocular pressure (IOP) of Spraque-Dawley rats receiving laser photocoagulation with that of a control group, with or without LBE2 treatment, in order to investigate the effect of LBE2 from Lycium barbarum on lowering IOP;
examining the weight and mortality of the animals, to investigate the possible acute and chronic toxicity of LBE2 on animals during normal condition, following optic nerve transection or in the ocular hypertensive model.

The dosages of the inventive LBE2 in the experiments described below ranged from 0.01 to 1000 mg/kg orally administered daily for Spraque-Dawley rats; and from 0.17 to 1700 mg/kg for hamsters.

Retinal ganglion cell (RGC) death associated with conditions such as glaucomatous optic neuropathy may be caused by more than one mechanism, including, but not limited to, excitotoxicity, reactive oxygen species-signaled or catalyzed reactions, or high intracellular calcium concentration. This invention showed that the extract of Lycium barbarum could effectively reduce RGC death from chronic and traumatic damage in mammals without any apparent reliance on the particular death mechanism.

Lycium barbarum is a famous Traditional Chinese herbal medicine which has functions of “nourishing the kidney and producing essence, nourishing the liver and brightening eyes”. It has been widely used as health-giving food for 2400 years. Extracts or active fractions of Lycium barbarum are particularly useful in the methods of the invention. “Lycium barbarum,” as used herein, refers to extracts of Lycium barbarum. It is also known as fructus lycii and Gou Qi Zi. A member of the botanical group Lycium is a substance that is capable of providing a similar physiological effect(s) as that provided by Gou Qi Zi in the compositions of the invention, and is preferably selected from a group comprising Fructus lycii; Lycium barbarum L.; Lycium chinense Mill.; Lycium turcomanicum Turcz; Lycium potaninii Pojank; Lycium dasystemum Pojank; Lycium europaeum (non L.); Lycium halimifolium (Mill.); Lycium lanceolatum (Veillard.); Lycium megistocarpum (Dun.); Lycium ovatum.; Lycium subglobosum.; Lycium trewianum. L. vu; Lycium europeum; Lycium halamifolium; Lycium halmifolium; Lycium vulgare.

The active agent of this invention is a hot water (i.e., above about 50° C.) extract which is substantially free of lower alcohol soluble components. The active agent is polyanionic, has a molecular weight of less than 500 kD and is insoluble in at least one of methylene dichloride, chloroform and toluene. It is preferably extracted from Lycium barbarum but may be present in other plants of Solanaceae. Based on the data set forth in the below examples, the preferred extract is a 50° C. to 100° C. water extract and most preferably, an about 70° C. extract.

The extract or active fraction of Lycium barbarum was isolated by the following procedure. First, the dried fruit of Lycium barbarum was extracted by ethanol resulting in an ethanol extract (LBE1). After separation and evaporation of the alcohol, the alcoholic extracted fruit was dried and contacted with hot water (70° C.) and the resulting extract was concentrated and dried as powder designated LBE2. The remaining residue was further extracted by boiling water (100° C.) resulting the powder LBE3. The remaining fruit residue was contacted with 5% NaOH overnight, followed by dialysis for 60 hours and desiccation; the resulting residue was designated LBE4.

The extracts of the invention are substantially free of lower (C₁₋₅) alcohol soluble material and constitute water soluble polysaccharides. LBE2 is the most preferred extract but any extract containing the active LBE2 can used although greater amounts may be necessary to achieve the same level of activity.

The type of glaucoma for which the invention is applicable includes but is not limited to: primary open angle glaucoma, normal pressure glaucoma, pigmentary glaucoma, pesudoexfoliation glaucoma, acute angle closesure glaucoma, absolute glaucoma, chronic glaucoma, congenital glaucoma, juvenile glaucoma, narrow angle glaucoma, chronic open angle glaucoma and simplex glaucoma. “Extract” as used herein, refers to the substances obtained from the specified source plant, or parts thereof (for e.g., fruit, root, bark, leaves). Any method of extraction that yields extracts that retain the biological activity of the substances contained in the extract source can be used to produce extracts used in this invention. Preferably, the ingredients of the compositions of the present invention are extracted as an aqueous solution. The extraction is preferably performed condition of normal pressure, and preferably at elevated temperatures (preferably within a range of 50° C. to 100° C., most preferably about 70° C.). The extract is preferably in a dried powder form. Concentration to powder form is preferably achieved by evaporation. It is understood that any method or conditions known in the art to yield extracts comparable in therapeutic effectiveness to those produced by the preceding preferred extraction method can be used for the purpose of this invention.

Furthermore, the term “extract” also refer to the active ingredients isolated from the fruit or other parts of Lycium barbarum or other natural sources including but not limited to all varieties, species, hybrids or genera of the plant regardless of the exact structure of the active ingredients, from or method of preparation or method of isolation. The term “extract” is also intended to encompass salts, complexes and/or derivatives of the extract which possess the above-described biological characteristics or therapeutic indication. The term “extract” is also intended to cover synthetically or biologically produced analogs and homologs with the same or similar characteristics yielding the same or similar biological effects of the present invention.

The purified composition contemplated for use herein include purified extract fractions having the properties described herein from any plant or species, preferably Lycium barbarum, in natural or in variant form, and from any source, whether natural, synthetic, or recombinant.

As used herein, “pharmaceutical composition” means a formulation containing therapeutically effective amounts of the compound or composition containing the extract of the invention as described above together with suitable diluents, preservatives, solubilizers, emulsifiers and/or carriers. The physical form of such a composition, i.e., solid, liquid, etc., is not limited By a “therapeutically effective amount” it is meant an amount of a Lycium barbarum extract that is sufficient to prevent and preserve RGC against degeneration or retard the degree of degeneration thereof. Of course, what constitutes a therapeutically effective amount will depend on a variety of factors, including, for example, the size, age, and condition of the subject, as well as on the mode of delivery. It is well within the ability of one of ordinary skill in the art to determine effective dosages.

Preferably, the subject is a human experiencing or at risk of developing a condition that is associated with RGC death, including glaucomatous optic neuropathy, ischemic optic neuropathy, inflammatory optic neuropathy, compressive optic neuropathy, and traumatic optic neuropathy. All of the above-mentioned conditions are associated with damage to the axonal region of RGC, as opposed to the cell body.

The Lycium barbarum extract will usually reduce RGC death by at least about 16%. However, it is expected a reduction of RGC death of only 10% or 5% will extend the vision of the treated subject. In a human subject, a reduction in retinal ganglion cell death may be estimated by extrapolation from functional and structural assays.

Functional assays involve evaluating changes in visual function over time, specifically, visual acuity and visual fields. It is reasonably concluded that a reduction in the rate of RGC death following initiation of Lycium barbarum extract treatment may be correlated with a reduction in the rate of loss of visual function over time. Structural assays involve visualizing or measuring the optic nerve head or the optic nerve fiber layer with an ophthalmoscope or other device to assess optic disc atrophy, disc cupping, or loss of nerve fibers.

In one preferred embodiment, a therapeutically effective amount of the Lycium barbarum extract will be administered topically to a human subject exhibiting symptoms of or at risk for developing a disease affecting retinal ganglion cells. Other modes of administration can also be used. The method of the present invention is preferably used to treat a subject with a disorder affecting the axons of retinal cell ganglion cells, including, but not limited to glaucomatous optic neuropathy, ischemic optic neuropathy, inflammatory optic neuropathy, compressive optic neuropathy, and traumatic optic neuropathy.

The pharmaceutically acceptable form of the composition includes a pharmaceutically acceptable carrier. Such pharmaceutically acceptable carriers are well known to those skilled in the art and are not limited. Additionally, such pharmaceutically acceptable carriers may be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or Axed oils. Intravenous vehicles include fluid and nutrient replenishers, electrolyte replenishers such as those based on Ringer's dextrose, and the like. Preservatives and other additives may also be present, such as, for example, antimicrobial, antioxidants, collating agents, inert gases and the like.

Controlled or sustained release compositions include formulation in lipophilic depots (e.g. fatty acids, waxes, oils). Also comprehended by the invention are particulate compositions coated with polymers (e.g. polyoxamers, polyoxamines, polyethylene glycol) and the extract coupled to antibodies directed against tissue-specific receptors, ligands or antigens or coupled to ligands of tissue-specific receptors. Other embodiments of the compositions of the invention incorporate particulates, protective coatings, protease inhibitors or permeation enhancers for various routes of administration, including parenteral, pulmonary, nasal and oral. Suitable excipients are, for example, water, saline, dextrose, glycerol, ethanol, or the like and combinations thereof. In addition, if desired, the composition can contain minor amounts of auxiliary substances such as wetting or emulsifying agents, pH buffering agents which enhance the effectiveness of the active ingredient.

The active extract can be formulated into the therapeutic composition as neutralized pharmaceutically acceptable salt forms. Pharmaceutically acceptable salts include the acid addition salts and which are formed with inorganic acids such as, for example, hydrochloric or phosphoric acids, or such organic acids as acetic, oxalic, tartaric, mandelic, and the like. Salts formed from the free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

In the therapeutic methods and compositions of the invention, a therapeutically effective dosage of the active component is provided. An amount based on patient characteristics (age, weight, sex, condition, complications, other diseases, etc.) will be selected, as is well known in the art. Furthermore, as further routine studies are conducted, more specific information will emerge regarding appropriate dosage levels for treatment of various conditions in various patients, and the ordinary skilled worker, considering the therapeutic context, age and general health of the recipient, is able to ascertain proper dosing. Generally, for intravenous injection or infusion, dosage may be lower than for intraperitoneal, intramuscular, or other route of administration. The dosing schedule may vary, depending on the circulation half-life, and the formulation used. The compositions are administered in a manner compatible with the dosage formulation in the therapeutically effective amount. Precise amounts of active ingredient required to be administered depend on the judgment of the practitioner and are peculiar to each individual. However, suitable dosages may range from about 0.01 mg/kg to 1700 mg/kg, more preferably about 0.01 to 1000 mg/kg, and most preferably about 10 mg/kg, of active ingredient per kilogram body weight of individual per day and depend on the route of administration. Suitable regimes for initial administration are also variable, and may be typified by an initial administration followed by repeated doses at one or more hour intervals by a subsequent injection or other administration. Alternatively, continuous intravenous, infusion sufficient to maintain concentrations of ten nanomolar to ten micromolar in the blood are contemplated.

The data provided in the Examples below demonstrate the invention. The examples below show that extract of Lycium barbarum protects axotomized RGC against cell death. Because axonal injury to RGC is implicated in most optic neuropathies, including glaucomatous optic neuropathy, inflammatory optic neuropathy, compressive optic neuropathy, and traumatic optic neuropathy, it is reasonably concluded that the RGC of an individual experiencing or at risk for developing one of these conditions will be protected by treating the individual with extract of Lycium barbarum.

The following examples are presented in order to more fully illustrate the preferred embodiments of the invention. They should in no way be construed, however, as limiting the broad scope of the invention.

### EXAMPLES

Animals

The experimental procedures conformed to the guidance of National Institutes of Health, U.S.A., for the care and use of laboratory animals. All efforts were made to minimize the number of animals used and their suffering.

### Example 1

### Effect of Lycium barbarum Extracts on Damages RGCs in an Optic Nerve Transection Model

The neuroprotective effect of four extracts isolated from Lycium barbarum was investigated. Adult hamsters (6-8 weeks, 60-80 g) were received optic nerve transection on their right eyes while their left eyes remained intact to serve as an internal control. All of the optic nerve were transected at 1.5 mm away from the optic disc. Immediately after injury, RGCs were labeled retrogradely by the use of a fluorescent dye called Fluorogold (FG). The RGCs in the left eyes were labeled 2 days before sacrificing the animals by the same method. By 7 days following the lesion, hamsters were euthanized under deep anesthesia (a lethal dose of sodium pentobarbitone). The retinae were fixed with 4% paraformaldehyde and were divided into four quadrants: superior, inferior, nasal and temporal. The whole retinae were then mounted on slides and examined under a fluorescent microscope. Labeled RGCs were counted along the median line of each quadrant starting from the optic disc to the peripheral border of the retina at 500 μm intervals, under an eyepiece grid of 200×200 μm. The percentages of surviving RGCs after different treatments were expressed by comparing the number of surviving RGCs in the injured eyes with their contralateral eyes.

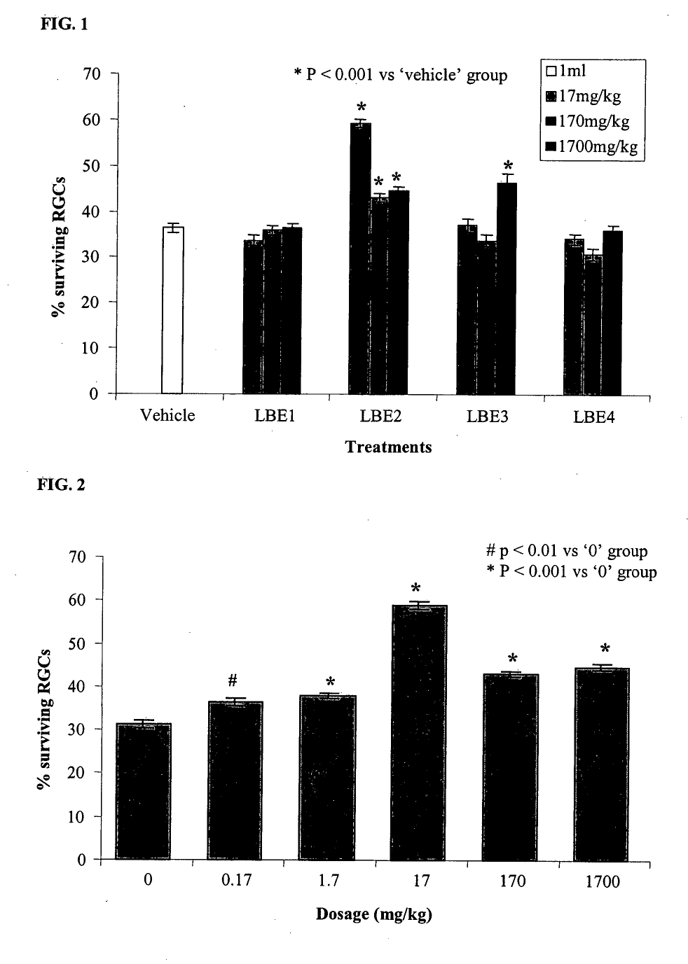

By 7 days following optic nerve transection, there were 36.3±0.92% surviving RGCs retained in the vehicle-treated control group (FIG. 1). All four extracts isolated from Lycium barbarum, namely LBE1, LBE2, LBE3 and LBE4 were tested at concentrations of 17, 170 and 1700 mg/kg body weight of subjects with 5 animals in each group. The subjects were fed with the extracts daily by a nasogastric tube from the first day of injury until euthanization. LBE1 did not promote the survival of RGCs following optic nerve transection at all concentrations being tested (17 mg/kg: 33.7±1.05%; 170 mg/kg: 35.9±0.95%; 1700 mg/kg: 36.4±1.0%). Treatment with 17 mg/kg LBE2 protected 59.0±0.88% of RGCs from damage while LBE2 at other concentrations tested promoted a lower percentage of RGCs to survive (170 mg/kg: 42.9±0.59%; 1700 mg/kg: 44.6±0.79%). Administration of lower concentration of LBE3 did not promote RGCs survival (17 mg/kg: 37±1.54%; 170 mg/kg: 33.5±1.14%) while LBE3 at 1700 mg/kg retained 46.3±1.98% RGCs on the retina following optic nerve transection. Treatment with LBE4 at any of the concentrations being tested did not protect RGCs from damage (17 mg/kg: 34.0±1.06%; 170 mg/kg: 30.8±1.15%; 1700 mg/kg: 35.8±1.13%). Adult hamsters were allowed to survive for 7 days after the lesion experiment. Among these four fractions, hamsters fed with LBE2 have the highest number of RGCs to survive. The data above is expressed as percentages of surviving RGCs compared with the total RGCs on the unoperated left eye (mean±SEM). Statistical significance was evaluated by one way ANOVA, followed by Bonferroni post hoc test. Differences were noted as significance for *p<0.001.

### Example 2

### Neuroprotection of Damaged RGCs by Administration of LBE2 after Optic Nerve Transection

Immediately following optic nerve transection, adult hamsters (6-8 weeks, 60-80 g) were fed with LBE2 daily (ranging from 0.17 to 1700 mg/kg) until euthanization (n=5 in each group). RGCs were labeled retrogradely with FG. Labeled RGCs were counted along each quadrant of retina (superior, inferior, nasal and temporal). In the control group of hamsters receiving vehicle, 31.3±0.87% of RGCs was retained 7 days following optic nerve transection (FIG. 2). In the group of hamsters receiving 17 mg/kg LBE2, the percentage of RGCs survival were increased to 59.0±0.88%. Increasing the concentration of LBE2 to 170 and 1700 mg/kg was not as effective as that of 17 mg/kg (42.9±0.59% and 44.6±0.79% respectively). Reducing the concentrations of LBE2 to 0.17 and 1.7 mg/kg did not elicit protective effects on RGCs from damage (0.17 mg/kg: 36.3±0.92%; 37.9±0.70%). Consequently, it was established that 17 mg/kg LBE2 could prevent 27.7% of RGCs to survive 7 days after optic nerve transection as compared with the control and therefore 17 mg/kg was considered the optimum dosage in this experiment. Adult hamsters (6-8 weeks, 60-80 g) were allowed to survive for 7 days after the lesion experiment. The data is expressed as percentages of surviving RGCs compared with the total RGCs on the unoperated left eye (mean±SEM). Statistical significance was evaluated by one way ANOVA, followed by Bonferroni post hoc test. Differences were noted as significance for #p<0.01 and *p<0.001.

### Example 3

### Neuroprotection of LBE2 on RGCs in an Ocular Hypertension Model of Spraque-Dawley Rats

The neuroprotective effect of different doses of LBE2 was investigated using an ocular hypertension model of Spraque-Dawley (SD) rats with 6 rats in each group. Adult SD rats (10-12 weeks, 250-280 g) were divided into different groups receiving vehicle (control) or LBE2 daily (ranging from 0.01 mg/kg to 1000 mg/kg) for 1 week. One week later, rats were received photocoagulation on their right eyes while their left eyes remained intact to serve as an internal control. Rats were continued to be fed with either vehicle or LBE2 until euthanization. Argon laser photocoagulation was applied to the limbal veins and 3 episcleral veins (2 superior and 1 inferior at the temporal area) under an operation microscope. In the first laser surgery, 120-140 laser spots (1000 μV, 0.1 s) were applied to the limbal and episcleral veins. To maintain an ocular hypertension, a second laser surgery with 60-120 spots was applied to block any vascular reconnection. The intraocular pressure (IOP) was measured to monitor the hypertensive condition by using a hand-held tonometer. IOP level was recorded before laser photocoagulation (as basal level) and 3 days after each laser photocoagulation (as post-operative IOP record).

Four days before sacrificing the animals, RGCs were retrogradely labeled with a fluorescent dye called Fluorogold (FG) and placed onto the superior colliculi of both sides of the midbrain of the rats. Rats were kept for 2 weeks after the first laser photocoagulation and euthanized with an overdose of anesthesia (ketamine and xylazine). The eyes were enucleated and fixed in 4% paraformaldehyde. The fixed eyeball was divided into two halves. The superior part included the optic disc and 2 mm optic nerve was left for further analysis. The inferior retinae were flat mounted onto gelatin-coated glass slides. FG-labeled RGCs were counted at 400× magnification. Seven adjacent areas (200×200 μm²) with each 500 μm separated (from optic disc to the peripheral) along each quadrant were taken. A total of 21 predefined fields were counted, which represented approximately 3.0 to 3.8% of the total retinal area (Laquis et al, 1998). FG-labeled RGCs showing condensed nuclei or fragmented nuclei were excluded (Nickells, 1999). Total number of living RGCs at the predefined areas in the injured eye was compared to that in the contralateral eye. The data is expressed in terms of relative percentage of FG-labeled RGCs loss in the injured eye to the contralateral intact eye, loss of FG-labeled RGCs (% contralateral, mean±SEM).

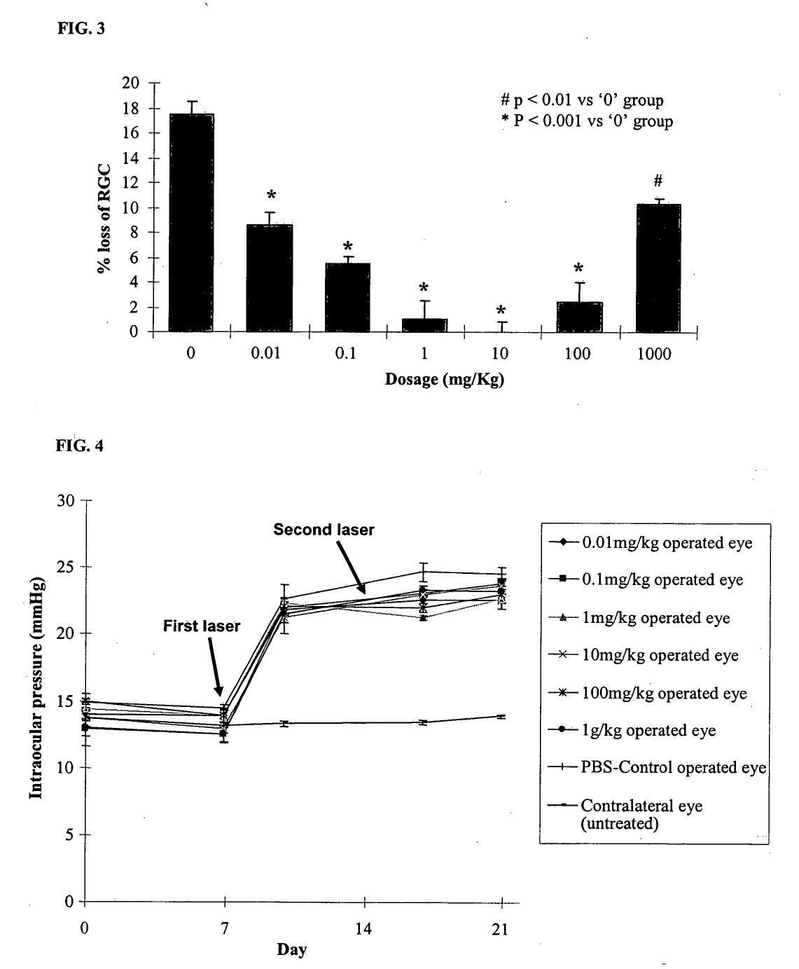

In the ocular hypertensive model, 17.0±1.1% of RGCs was lost in the injured eyes of the vehicle-treated control rats 2 weeks following the lesion (FIG. 3). Daily treatment of the LBE2 at either 0.01, 0.1, 1, 10, 100 or 1000 mg/kg showed neuroprotection on RGCs survival. No RGCs loss was detected in rats after feeding the rats with 10 mg/kg LBE2 (0±0.9%, p<0.001 compared to control). LBE2 at concentration of 1 mg/kg or 100 mg/kg also protected all RGCs from damage induced by laser photocoagulation (1 mg/kg: 1.0±1.6%, *p<0.001; 100 mg/kg: 2.4±1.7%, p<0.001 compared to control). There was no significant difference among rats fed with 1, 10 or 100 mg/kg of LBE2. Treatment with other concentrations of LBE2 was not as effective as the dosages mentioned above in protecting RGCs from ocular hypertension (0.01 mg/kg: 8.6±1.1%; 0.1 mg/kg: 5.5±0.5%; 1000 mg/kg: 10.3±0.55%). From these results, 1-10 mg/kg LBE2 were considered as the optimum dosages in this experiment. Data is expressed as percentage of RGCs loss compared with the total RGCs on the unoperated left eye (mean±SEM). #p<0.01 and *p<0.001 was noted as significance after one-way ANOVA followed by Bonferroni multiple comparisons test.

### Example 4

### Change in Intraocular Pressure (IOP) in an Ocular Hypertensive Model of Spraque-Dawley Rats

Adult Spraque-Dawley rats (10-12 weeks, 250-280 g) were induced to have ocular hypertension by laser photocoagulation. In all groups, the right eyes of the rats were photocoagulated by argon laser while their left eyes were unoperated as the contralateral control. Argon laser photocoagulation was applied to the limbal veins and 3 episcleral veins (2 superior and 1 inferior). Rats were fed daily with either vehicle (control) or LBE2 at concentrations ranging from 0.01 mg/kg to 1000 mg/kg one week prior to the laser photocoagulation until sacrifice (n=6 in each group). The intraocular pressure (IOP) was measured by a hand-held tonometer before laser photocoagulation and 3 days after each laser operation to monitor their hypertensive condition. Before laser application, the basal IOP of both eyes was 14.6±0.4 mmHg (FIG. 4). Ocular hypertension was induced by two laser applications, one on day 7 and one on day 14. Rats were euthanized on day 21. The level of IOP of the injured eyes remained at high level (22.6±1.1 mmHg to 24.6±0.7 mmHg) after the two laser applications. The ocular hypertension was therefore retained and was about 1.7-fold higher than that of the contralateral (intact) eyes. Elevated IOP was still retained in the groups of rats treated with 0.01, 0.1, 1, 10, 100 or 1000 mg/kg LBE2, ranging from 20.8±0.3 mmHg to 26.3±1.2 mmHg. The increased IOP level was as high as that of the vehicle-treated control rats. These results demonstrated that the LBE2 treatment did not reduce the elevated IOP. Treatment of laser photocoagulation induced an increase in intraocular pressure up to about 20 mmHg. While rats fed with LBE2 showed a reduction in RGCs loss, high IOP was not altered by LBE2. Data were evaluated by one way ANOVA, followed by Tukey-Kramer post hoc test.

### Example 5

### Chronic Toxicity Test of LBE2 in Hamsters with Optic Nerve Transection

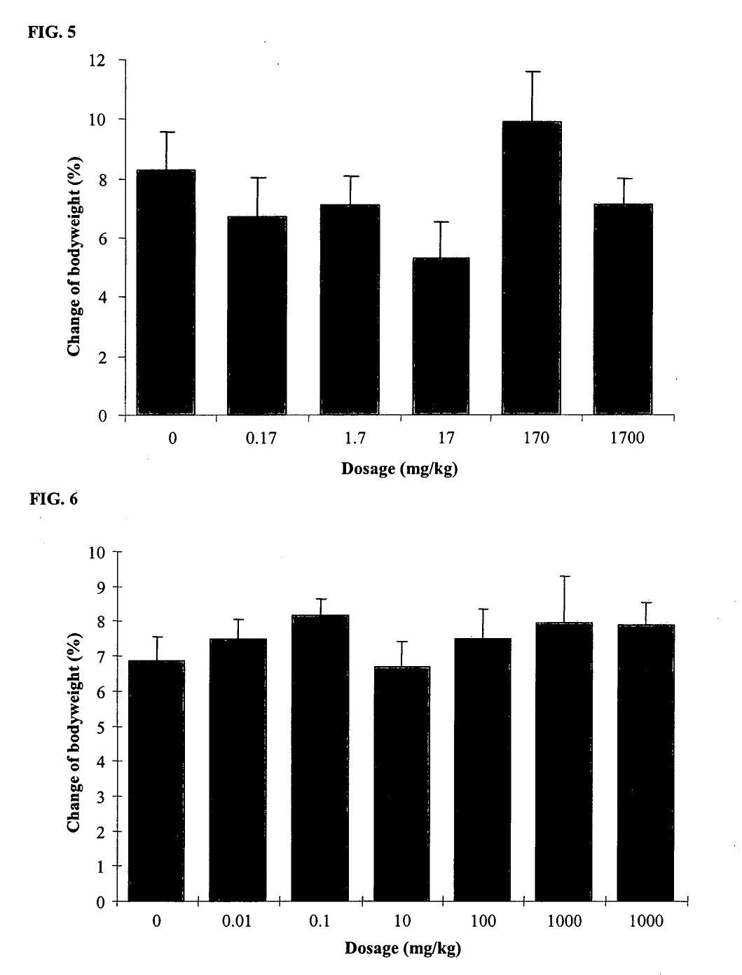

To investigate the possible chronic toxicity of LBE2 on adult hamsters (6-8 weeks, 60-80 g) in the optic nerve transection model, the body weight and mortality of the hamsters were recorded before and 7 days after optic nerve transection (n=8 in each group). Data are expressed as the change in body weight before and 7 days after the lesion (FIG. 5). Since the body weight of hamsters receiving different dosages of LBE2 were not affected at any concentrations being tested and never led to death, LBE2 is not considered to exert any chronic toxicity. The data was obtained from 3 independent experiments.

### Example 6

### Chronic Toxicity Test of LBE2 in an Ocular Hypertensive Model of Spraque-Dawley Rats

In adult Spraque-Dawley rats (10-12 weeks, 250-280 g) receiving LBE2 orally from 7 days before the induction of ocular hypertension to 14 days afterwards, the body weight and mortality of the rats were recorded to examine the possible chronic toxicity of LBE2 (n=6 in each group). The increase in body weight of the rats fed with LBE2 was similar to that of the vehicle-treated control rats (FIG. 6). The mortality of the LBE2 treatment was not significantly different compared to that in the control group. This demonstrated that oral consumption of LBE2 has no apparent side effects. Having fed with LBE2 and received laser photocoagulation, the rats were kept for 2 weeks and the body weight of rats were measured before euthanization. From the results, the body weight of rats fed with LBE2 were not affected.

### Example 7

### Chronic Toxicity Test of LBE2 in Normal Young Spraque-Dawley Rats

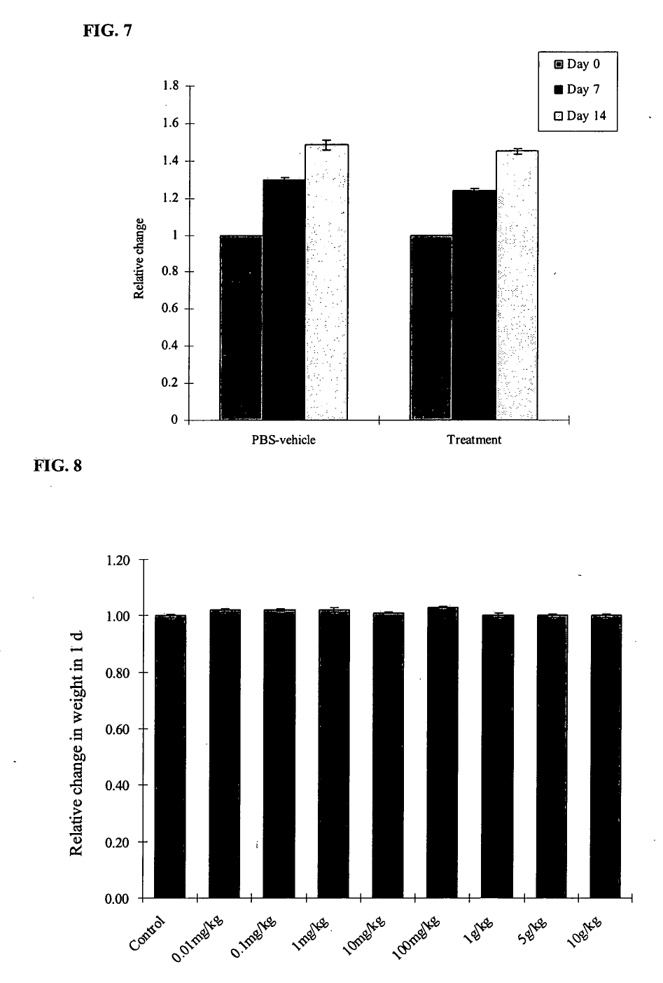

To investigate whether the LBE2 demonstrate any toxic effect in normal young Spraque-Dawley rats (4 weeks), rats were fed with 10 g/kg of LBE2 for 2 weeks (n=8 in each group). The change of body weight of rats before and after oral administration of LBE2 and also the number of rats which died were recorded. Data were obtained from 3 independent experiments. The results (FIG. 7) show that there was no significant change of body weight of rats fed with the extract, suggesting no chronic toxicity.

### Example 8

### Acute Toxicity Test of LBE2 in Normal Spraque-Dawley Rats

Acute toxicity for the LBE2 treatment was investigated by using the LD50 value as the standard. Normal adult Spraque-Dawley rats (10-12 weeks, 250-280 g) were divided into groups receiving either vehicle or different dosages of LBE2 (0.01 mg/kg, 1000 mg/kg and 10000 mg/kg). By 24 hours after oral administration, the number of dead rats was counted in each group. The results (FIG. 8) show that there was no significant change of body weight of rats fed with the extract and no mortality was recorded, suggesting no acute toxicity.

Various changes and modifications can be made to the present invention without departing from the spirit and scope thereof. The embodiments illustrated were for further exemplification of the invention and were not intended to limit it.

### REFERENCES

The full citation of the references cited above, and incorporated herein by reference are:
Cheung Z H, So K F, Lu Q, Yip H K , Wu W, Shan J J, Pang P K and Chen C F (2002).

Enhanced survival and regeneration of axotomized retinal ganglion cells by a mixture of herbal extracts. J Neurotrauma 19: 369-78.
Chinese Herbal Medicine Company (1994). Chinese Traditional Formulation of Herbal Medicine, 1598-1608 Scientific Publication Shanghai, China.
Cho K S, Chan P M, So K F, Yip H K and Chung S K (1999). Ciliary neurotrophic factor promotes the regrowth capacity but not the survival of intraorbitally axotomized retinal ganglion cells in adult hamsters. Neuroscience 94: 623-8.
Cho K S, Chung S K, Yip H K and So K F (2001). Differential effects of intravitreal optic nerve and sciatic nerve grafts on the survival of retinal ganglion cells and the regeneration of their axons. J Neurocytol 30: 983-91.
Frost D O, So K F and Schneider G E (1979). Postnatal development of retinal projections in Syrian hamsters: a study using autoradiographic and anterograde degeneration techniques. Neuroscience 4: 1649-77.
Garcia-Valenzuela E, Shareef S, Walsh J and Sharma S (1995). Programmed cell death of retinal ganglion cells during experimental glaucoma. Exp Eye Res 61:33-44.
Ji J Z, Elyaman W, Yip H K, Lee V W, Yick L W, Hugon J and So K F (2004). CNTF promotes survival of retinal ganglion cells after induction of ocular hypertension in rats: the possible involvement of STAT3 pathway. Eur J Neurosci 19: 265-272.
Lam K W and But P (1999). The content of zeaxanthin in Gou QI Zi, a potential health benefit to improve visual acuity. Food Chem 67:173-176.
Laquis S, Chaudhary P and Sharma S (1998). The patterns of retinal ganglion cell death in hypertensive eyes. Brain Res 784:100-104.
Laquis S, Chaudhary P and Sharma S C (1998). The patterns of retinal ganglion cell death in hypertensive eyes. Brain Res 784:100-4.
Leske M C (1983). The epidemiology of open-angle glaucoma: a review. The epidemiology of open-angle glaucoma: a review. Am J Epidemiol 118:166-91.
Lu Q, Cui Q, Yip H K and So K F (2003). c-Jun expression in surviving and regenerating retinal ganglion cells: effects of intravitreal neurotrophic supply. Invest Ophthalmol Vis Sci 44: 5342-8.
McKinnon S J, Lehman D M, Tahzib N G, Ransom N L, Reitsamer H A, Liston P, LaCasse E, Li Q, Korneluk R G and Hauswirth W W (2002). Baculoviral IAP repeat-containing-4 protects optic nerve axons in a rat glaucoma model. Mol Ther 5: 780-7.
Mittag T W, Danias J, Pohorenec G, Yuan H M, Burakgazi E, Chalmers-Redman R, Podos S M and Tatton W G (2000) Retinal damage after 3 to 4 months of elevated intraocular pressure in a rat glaucoma model. Invest Ophthalmol Vis Sci 41:3451-3459.
Morrison J, Moore C, Deppmeier L, Gold B, Meshul C and Johnson E (1997). A rat model of chronic pressure-induced optic nerve damage. Exp Eye Res 64:85-96.
Nickells R W (1999). Apoptosis of retinal ganglion cells in glaucoma: an update of the molecular pathways involved in cell death. Surv Ophthalmol 43 Suppl 1:S151-61.
Osborne N N, Wood J P, Chidlow G, Bae J H, Melena J and Nash M S (1999). Ganglion cell death in glaucoma: what do we really know? Br J Ophthalmol 83:980-6.
Quigley H A and Green W R (1979). The histology of human glaucoma cupping and optic nerve damage: clinicopathologic correlation in 21 eyes. Ophthalmology 86:1803-30.
Quigley H A and Pease M E (1996). Change in the optic disc and nerve fiber layer estimated with the glaucoma-scope in monkey eyes. J Glaucoma 5:106-16.
Quigley H A (1996). Number of people with glaucoma worldwide. Br J Ophthalmol 80:389-93.
Sawada A and Neufeld A (1999). Confirmation of the rat model of chronic moderately elevated intraocular pressure. Exp Eye Res 69:525-531
So K F, Campbell G and Lieberman A R (1985). Synaptic organization of the dorsal lateral geniculate nucleus in the adult hamster. An electron microscope study using degeneration and horseradish peroxidase tracing techniques. Anat Embryol (Berl) 171: 223-34.
So K F, Schneider G E and Frost D O (1978). Postnatal development of retinal projections to the lateral geniculate body in Syrian hamsters. Brain Res 142: 343-52.
Ueda J, Sawaguchi S, Hanyu T, Yaoeda K, Fukuchi T, Abe H and Ozawa H (1998). Experimental glaucoma model in the rat induced by laser trabecular photocoagulation after an intracameral injection of India ink. Jpn J Ophthalmol 42:337-344.
WoldeMussie E, Ruiz G, Wijono M and Wheeler L A (2001). Neuroprotection of retinal ganglion cells by brimonidine in rats with laser-induced chronic ocular hypertension. Invest Ophthalmol Vis Sci 42: 2849-55.
WoldeMussie E, Yoles E, Schwartz M, Ruiz G and Wheeler L A (2002). Neuroprotective effect of memantine in different retinal injury models in rats. J Glaucoma 11: 474-80.
Woo H H, Jen L S and So K F (1985). The postnatal development of retinocollicular projections in normal hamsters and in hamsters following neonatal monocular enucleation: a horseradish peroxidase tracing study. Brain Res 352: 1-13.
You S W, Bedi K S, Yip H K and So K F (2002). Axonal regeneration of retinal ganglion cells after optic nerve pre-lesions and attachment of normal or pre-degenerated peripheral nerve grafts. Vis Neurosci 19: 661-8.

1. A method for reducing retinal ganglion cells death in a subject comprising administering an agent (LBE) extracted by water from a Lycium barbarum -containing material to the subject.

2. The method of claim 1, wherein said LBE is an extract of a Solanaceae plant.

3. The method of claim 1, wherein said LBE is a hot water extract of Lycium barbarum substantially free of lower alcohol extractable components.

4. The method of claim 1, wherein said LBE is polyanionic, has a molecular weight of less than 500 kD and is insoluble in at least one of methylene dichloride, chloroform and toluene.

5. The method of claim 2, wherein said LBE is a 50° C. to 100° C. water extract of Lycium barbarum substantially free of lower alcohol extractable components.

6. The method of claim 2, wherein said LBE is about 70° C. water extract of Lycium barbarum substantially free of lower alcohol extractable components.

7. The method of claim 1, wherein the agent is administered in combination with a pharmaceutically acceptable carrier.

8. The method of claim 1, wherein said administration is intravenously, intracranially, intracerebrally, subcutaneously, intramuscularly, intranasally or intraperitoneally.

9. The method of claim 1, wherein said subject is human.

10. The method of claim 1, wherein said administration is daily.

11. The method of claim 1, wherein said administration is oral.

12. The method of claim 1, wherein said administration is topical.

13. The method of claim 1, wherein the subject is a human having or suspected of having glaucoma.

14. The method of claim 13, wherein said LBE is a 50° C. to 100° C. water extract of Lycium barbarum substantially free of lower alcohol extractable components.

15. The method of claim 14, wherein said LBE is administered in combination with a pharmaceutically acceptable carrier.

16. The method of claim 15, wherein said administration is oral or topical.

17. The method of claim 16, wherein the amount of LBE administration is about 0.01 to 1700 mg/kg.

18. The method of claim 13, wherein said LBE is about 70° C. water extract of Lycium barbarum substantially free of lower alcohol extractable components.

19. The method of claim 18, wherein said LBE is administered in combination with a pharmaceutically acceptable carrier.

20. The method of claim 19, wherein said administration is oral or topical.
